(19) United States
(12) Patent Application Publication
(10) Pub. No.: US 2004/0241239 A1
(43) Pub. Date: Dec. 2, 2004

(54) Mixed complexes for masking the taste of bitter active substances

(76) Inventor: Petra Bastian, Regensburg (DE)

Correspondence Address: NIXON & VANDERHYE, PC, 1100 N GLEBE ROAD, 8TH FLOOR, ARLINGTON, VA, 22201-4714, US

(21) Appl. No.: 10/483,405

(22) PCT Filed: Jul. 11, 2002
(86) PCT No.: PCT/EP02/07759

(30) Foreign Application Priority Data
Jul. 11, 2001 (DE) 10133546.6

(51) Int. Cl. A61K 31/7048; A61K 9/14

(52) U.S. Cl. 424/486; 424/488; 514/29

(57) ABSTRACT

The subject of the invention is an at least a ternary ionic complex with a taste that is not unpleasant, which comprises the following components:

a) at least one unpleasantly tasting active substance which bears at least one ionizable cationic group.

b) at least one negatively charged polymer which comprises at least one anionic group and

c) at least one positively charged polymer, which comprises at least one cationic group.

This complex does not show an unpleasant taste, either in solution or upon ingestion. The active substance is released upon action of the stomach-intestinal juices.

The subject of the invention is a method for improving the taste of unpleasant, particularly bitter-tasting active substances, which bear at least one cationic center, as well as the non-bitter-tasting complex obtained by this method.

The masking of a bad or bitter taste of active substances is a recurring problem in the professional literature. Thus, for a long time, envelopes for bitter-tasting preparations have been described, wherein sugar, polymers or other materials have been used for producing the envelopes.

In the case of production of liquid preparations, which can be administered more easily even to children or persons with swallowing difficulties, essentially two methods are described which are not based on a chemical modification of the active substance. The first involves the production of an enveloped or embedded particulate granulate, and the second method involves the complexing of the active substance ionically.

The objective of both approaches is to prevent the presentation of the active substances to the taste receptors of the tongue. For this purpose, formulations must be found, which can be dispersed in the aqueous phase without leading to a release or presentation of the active substance. If an envelope or embedding of the active substance is used, however, this usually has the consequence that also in the gastrointestinal tract, where the active substance is to be released, there will be a delayed release and thus there will be a reduction in its bioavailability.

The masking of the active substance based on ionic interactions, named “complexing” here, is described in many references. Examples therefor include the complexing of active substances with anionic polymers. Thus, the complexing of the antibiotics erythromycin and clarithromycin with polyacrylic acid derivatives (Carbopols and Carbomers) has been described in the literature (Fu Lu, M.-Y., A polmer carrier system for taste masking of macrolide antibiotics, Pharm. Res. 8(6) (1991), 706-712 and U.S. Pat. No. 4,808,411; EP 0 943,341; WO 97-16174; EP 293,885; U.S. Pat. No. 3,346,449) as well as the complexing of bufomedil, a vasodilatory substance, with an acrylic acid derivative and a cellulose ether derivative (WO 94-27,596).

The problem in the case of these binary complexes, however, is that the masking of taste is usually insufficient, so that for the masking of the bitter taste, an additional envelope is necessary (Fu Lu, M.-Y., A polymer carrier system for taste masking of macrolide antibiotics, Pharm. Res. 8(6) (1991), 706-712 and EP 0 943,341; WO 94-27,596).

Possibly, the ionic complexing of active substances containing cationic groups with polymers containing anionic groups is insufficient for the masking of the bitter taste, since the anionic groups are arranged in a sterically unfavorable manner in the polymer. For example, the carboxyl groups in polyacrylate lie close together, whereas most active substances, such as macrolide antibiotics, involve non-straight, small molecules, and the cationic groups to be saturated and neutralized are difficult to access due to steric hindrance. It is thus well conceivable that with an anionic polymer, such as polyacrylate or carboxymethylcellulose, the cationic groups are hard to saturate with such active substances, i. e. It does not result in 1:1 complexes relative to charge density. The unsaturated ionic groups that remain possibly endow the complex with a residual water solubility, which leads to the fact that the molecules of active substance and polymer molecules can be shifted in the gel and thus the active substance—which is bound or free —can be presented to the taste receptors.

It is also described in the literature that anionic polymers, which are used for complexing, may also be used for the production of a coating for the active substance. However, this is associated in turn with the problem of reduced bioavailability (Friend, D., Polyacrylate resin microcapsules for taste masking of antibiotics, J. Microencapsulation, 9 (1992), 469-480).

It is thus clear that the masking of suspensions for oral administration, which has been described previously in the literature, is not sufficient; rather, in the described binary complexes of active substances with anionic polymers, the active substance will be released too early, or the active substance in the binary complex will be presented, just as it was before, with the generation of the undesired taste; the unpleasant taste is thus not sufficiently masked. The unpleasant taste can then be masked by an envelope, but this leads to the delay of the release time and thus to the reduction of bioavailability (Friend, D., Polyacrylate resin microcapsules for taste masking of antibiotics, J. Microencapsulation, 9 (1992), 469-480).

WO 00/05269 describes polymer complexes, which are comprised of a polysaccharide containing glucuronic acid, a cationic polymer which must not be a protein, as well as an active substance, as the case may be. The complexes formed thereby are precipitated, however, by the addition of alcoholic solvents, so that obviously water-soluble and thus bitter-tasting complexes were obtained.

The object of the present invention was thus to develop a form of administration for an unpleasant, particularly bitter-tasting active substance, in which the unpleasant taste of the active substance is masked, without a reduction in the bioavailablity of the active substance by embedding or enveloping.

The object is solved according to the invention by providing a water-insoluble complex, which is at least ternary and which comprises the following compounds:

a) at least one bitter-tasting, active-substance molecule with at least one cationic group

b) a substance, an oligomer or polymer with at least one anionic group and

c) a substance, an oligomer or polymer with at least one cationic group.

It was surprisingly established that complexes, which are formed with the use of components a), b) and c) used according to the invention, can be obtained in water-insoluble form. This insolubility in water is characterized, for example, by a spontaneous precipitation in aqueous media without addition of non-solvents for the complex, for example, organic solvents. The insolubility in water or a spontaneous precipitation, which leads to solid suspended particles is essential in order to effectively mask the bitter taste of an active-substance molecule.

Discrete particles with a diameter in the millimeter to micrometer range, particularly particles with a diameter ≧300 nm, preferably ≧1 μm, more preferably ≧10 μm and most preferably ≧50 μm are preferably formed according to the invention in the case of spontaneous precipitation.

It was established according to the invention that the bitterness of active substances in complexes can still be perceived if at least a trace of water solubility is still present. In complexes which are not completely insoluble in water, a gel is often formed instead of a precipitate. In the presence of a gel, the active substance is still presented to the taste receptors in the mouth and this leads to a bitter taste.

Particularly advantageous results with respect to water insolubility or spontaneous precipitation can be obtained by optimizing the individual components with respect to the steric conditions. The solubility of molecules in solvents, such as, e.g. in water is mediated by the hydrophilic/lipophilic factor or by hydrophilic/lipophilic molecular fractions, or by polar/non-polar or polarizable or ionic or ionizable molecular fractions.

The solubility in water as a solvent presumes polar, polarizable or ionic groups, and thus an interaction can result with the dipole water by hydrogen bridges. Interface energy and lattice energy are overcome only in this way.

Ions (charge carriers) in principle are present in water in a well-dissolved state. In general, in addition to an ion, its counterion is also present and dissociated in water. Only if the dissociation constant (an equilibrium constant) is exceeded, will a specific fraction of a solid salt of anions and cations, in addition to the dissolved fraction, be present in the solution.

The dissociation constant of salts is variable. In principle, large ions form hardly soluble salts with large counterions. However, it is essential for water insolubility that the total charge of the ion is saturated by the one or more counterions. This means that the obtaining of water solubility generally will no longer predominate opposite lipophilia, if all charges of an ion are neutralized by charges of a counterion.

In the case of hydrophilic polymers, whose hydrophilic nature is obtained, e.g. by a large quantity of carboxyl groups, a hardly soluble ion complex can be formed with cationic molecules. Of course, the density of the hydrophilic groups is usually provided by the polymer. Often polymers show a high regularity and a great density of negatively charged groups, such as as carboxyl groups (in particular, polyacrylates). Most pharmaceutical substances, which should now desirably form the counterion, are often relatively large (compared with sulfate, tetramethylammonium, etc.). Thus their size prevents the fact that all negative charges, e.g., all carboxyl groups, will be saturated, since these are too dense (steric hindrance). Negative charges remain, e.g., in the form of carboxyl groups which cannot form a complex and are insufficiently screened so as not to cause solubility in water. Therefore, such complexes previously were insufficient in their complexing. If the complex only has subregions with free carboxyl groups, often a turbid gel is formed, which never precipitates from the solution in a completely anhydrous manner. For drying, either desolvation with organic solvents must be conducted, or the residual water must be withdrawn by lyophilization, drying or by other methods. The product, however, will always absorb water and form a gel. A gel, which contains water, however, has the disadvantage that the molecules are present in a flexible and movable state and in this way a presentation to the taste receptors of the active substance bound in the gel is also possible.

Only a complete saturation of the negative charges, e.g., of the carboxyl groups can lead to a compound that no longer bears water solubility in the molecule. It is in turn advantageous to select the saturating or neutralizing ions in a correspondingly large size in order to produce an appropriate dissociation constant. Sodium and potassium ions are too small. Also, divalent calcium is not suitable in this case. As the smallest molecule that can bring the complex according to the invention to precipitation, trimethylammonium is suitable.

However, other small cations, such as, e.g., tetraphenylphosphonium ions, may also be used. Smaller cations are advantageously used for cross-linked polymers, since they can reach into smaller gaps. Larger cations, particularly cationic polymers, however, are preferred due to their greater physiological acceptance for the most part in many applications.

It is important here to distinquish between a precipitation of the polymers by calcium and a precipitation of the complex with the active substance. Calcuim precipitates polymers that contain carboxyl groups in that it combines chains/carboxyl groups with one another via the divalent charge. In this way, the size of the molecule increases in the complex and it precipitates. Any type of steric hindrance, however, reduces the complexing of the polymer with calcuim, since a competition ensues. On the one hand, calcium is too small to saturate individual carboxyl groups in the polymer, and on the other hand, one positive charge is left over. Calcium will thus lose its ability to compete for precipitation in any case in comparison to a large molecule. Logically, the complex of polymer and active substance cannot be made insoluble in water with calcuim.

In principle, it is meaningful and preferred to optimize the complex so that a precipitation is produced by ions that are specifically suitable for [filling] the gaps between polymer and active substance. In this way, the active substance is simultaneously better screened from being presented to the taste receptors. This is indicated by milk protein, for example. Milk protein consists of linear, flexible peptide and protein chains, which may have charge carriers at different places. The flexible chains can be incorporated into the intermediate spaces and the remaining parts that project out can screen the active-substance molecules.

Accessible molecules are preferably utilized as much as possible as components. It has thus been emphasized that cross-linking may be troublesome in the case of specific receptors. If, for example, milk protein is used as a “spacer”, then the molecules are too large to be effective in a three-dimensional network. The steric hindrance here makes the “gaps” inaccessible to the “spacer”. The consequence is that the complex cannot be precipitated. The degree of cross-linking, which is troublesome here, may be readily specified appropriately for the components concerned by the person of average skill in the art. It is dependent both on the size of the active-substance molecule (for example, it is known that large proteins cannot be inserted into greatly cross-linked polymers) as well as on the spacer, which must still provide access to the “gaps” after complexing with the active substance (the largest molecule “precipitates” first) (calculation via molecule size/charge number). Smaller molecules, such as, e.g., the trimethylammonium ion, reach the gaps in any complex, as long as the active substance is deposited.

It is thus possible according to the invention to adapt the components sterically to one another so that a complex that is insoluble in water is formed.

The complexes according to the invention are further characterized in that they are insoluble in water, but under the conditions in the gastrointestinal tract, particularly in the stomach or the intestine, will rapidly decompose. This supplies the advantage that the bioavailability of the active substance in the complex is not adversely affected. Correspondingly, according to the invention, proteins may also be used as the positively charged component (component c), since proteases reinforce the rapid decomposition of such complexes in the gastrointestinal tract.

According to the invention, a water-insoluble complex is obtained by combining at least three components. Water insolubility in the sense of the invention means particularly that the complex precipitates spontaneously from an aqueous solution. Preferably, water insoluble means that less than 1 mg of the complex remains in solution in 30 ml of pure water, particularly less than 1 mg of the complex remains in solution in 100 ml, particularly in 1000 ml of pure water.

The complex is further characterized in that it does not have a bitter taste. As will be explained in detail herein, the water insolubility of the total complex results from the fact that bitter components of the complex are not available for uptake by the taste receptors and thus a bitter taste cannot be perceived. The grading of bitterness can be determined, e.g., according to conventional organoleptic tests. The organoleptic test for establishing bitterness can be conducted according to the usual procedures, e.g., as in the DAB [German Pharmacopeia] under 2.8N8. Preferably, the solution over the complex according to the invention tastes just like water. In addition, a bitter taste develops slowly, if one places a particle of a complex according to the invention in his mouth so that it remains in the mouth at least 10, more preferably 15 minutes, and most preferably at least 20 to 30 minutes.

Component a) of the ternary water-insoluble complex is a bitter or unpleasantly tasting active-substance molecule. This active-substance molecule is characterized in the presence of at least one cationic group. A cationic group is thus a group, which is positively charged or can be protonated under normal physiological conditions. Examples of such cationic groups are primary, secondary, or tertiary amines, also [the amines] in amino acids, quaternary amines, metals in organometallic compounds, e.g., platinum, antimony, palladium, cobalt and other metal compounds, phosphonium compounds, oxonium compounds and others.

The cationic group of the bitter active-substance molecule according to (a) preferably is a protonatable amine.

The active-substance molecule according to (a) is preferably a bitter active substance, for example, an alkaoid or cardiac glycoside or a hormone or a diuretic or a CNS active substance or a medication preventing inflammation or a pain medication or a cytostatic or a liver therapuetic or antihistamine or a corticosteriod or an interferon or an antibiotic, especially preferably erythromycin, clarithromycin or HMR 3647.

As a second component, the complex according to the invention contains a substance, an oligomer or/and a polymer with at least one anionic group. An anionic group under physiological conditions has one negative charge or can be deprotonated. Component (b), due to its opposite charge, can form a complex with component (a) and this complex is bonded by electrostatic interactions. Component (b) thus serves as the counterion for the charge of the active-substance molecule.

The anionic group according to (b) preferably is an acid function, particularly a carboxylic acid group or an acid function of phosphorus, nitrogen or sulfur. . . . * be an oligomer, particularly an oligomer formed from 2-20 monomer units or/and a polymer. The polymer according to (b) preferably is a cellulose derivative, particularly carboxymethylcellulose or polyacrylic acid or polymethacrylic acid or acid-substituted siloxanes or silicones, or acid-substituded resins, particularly acidic epoxy resins, or acid-substituted dextrans or acid-substituted chitosan, or acid-substituted polystyrenes, or peptides or DNA or RNA or oligonucleotides.

Finally, a component with at least one cationic group is utilized as component (c). This compound serves for the purpose of saturating left-over charges that are possibly present in the complex of (a) and (b) and thus provides the desired insolubility in water. Component (c) may be a low-molecular substance, for example, lecithin, an oligomer or/and a polymer. Other preferred low-molecular substances are trimethylammonium, tetramethylammonium, tetraethylammonium, tetrabutylammonium, tetraphenylphosphonium and others. Component (c) is preferably a molecule, whose size (volume) is greater than or equal to that of trimethylammonium.

The polymer with the basic ionizable group according to (c) preferably is a micelle-forming polymer and/or a protein or a peptide or a polysaccharide, particularly chitosan, polylysine or a mixture of proteins or peptides, particularly soy or milk products and/or resins, particularly epoxy resins, which are substituted with basic groups, and/or polystyrenes, which are substituted with basic groups, and/or dextrans, which are substituted with basic groups. In particular, a polymer with a high polydispersability is utilized as component c). The use of milk proteins, particularly of basic fractions of milk proteins, is particularly preferred.

A polymer with a high polydispersability is utilized as component (c). The use of milk proteins is most preferred.

A “de-bittering” of proteins of milk or soy products has already been described in the literature. Thus Tamura (Tamura, M., Miyoshi T., Mori N., Kinomura, K, Kawaguchi, M., M. Ishibashi N., Okai H., Mechanism for the bitter tasting potency of peptides using O-aminoacyl sugars as model compounds, Agric. Biol. Chem., 54 (6) (1990), 1401-1409)) describe that milk and soy products can be used in order to mask the taste of bitter-tasting peptides.

However, most combinations of bitter substances with additives to provide mildness only have a small capacity, which is generally insufficient for practical application. As determined in our own investigations, one achieves only an approximately 3-fold increase in the limiting concentration with the addition of milk powder to a solution of active substance, which is insufficient for practical application. The direct complexing of active substances with milk or soy products is accordingly unsuitable. It has now been found that proteins and peptides, particularly of milk and soy products, can be utilized in a meaningful way in order to obtain application forms with the desired physicochemical properties that mask taste only after formation of a complex of the cationic active substance with anionic polymers.

The “de-bittering” effect of milk and soy products is possibly attributed to the fact that the unsaturated partial charges of the polymer can be saturated by the molecules that are used, whereby, for example, these are flexible proteins or peptides. In this way, the existing complex is stabilized and a non-bitter precipitate is formed. This is dissolved relatively easily in the gastrointestinal tract due to ionic interactions. If proteins or peptides are used for the formation of the complex, which is at least ternary, then these proteins or peptides will also be digested by proteases or peptidases. Thus a rapid dissolution of the complex in the digestive juices is assured and the problem of reduced bioavailability is solved.

According to the invention, it is thus possible to prepare active substances in a form, which has no bitter taste in the mouth, but is rapidly made available due to its ready dissolution in the gastrointestinal tract. The complex of active substance according to the invention is stable in pure water at pH 3-9, particularly 3-4, for at least 1 hour, in particular for at least 2 hours, more preferably for at least 5 hours. A decomposition occurs as soon as the complex of active substance is subjected to conditions as are present in the gastrointestinal tract, thus particularly a reduced pH, for example, a pH ≦2.5, particularly a pH ≦2.

The complex of active substance according to the invention preferably decomposes in concentrated hydrochloric acid and is stable at pH 3-4 for at least several hours in water. The complex of active substance is further preferably characterized in that the fraction of active substance is greater than 40%, particularly greater than 50%. It is possible according to the invention to produce complexes of active substance with a high proportion of active substance.

The complexes of active substance according to the invention are provided particularly for peroral administration. The masking of bitterness is of particular importance in peroral administration. Another subject of the invention is thus a pharmaceutical containing a water-insoluble complex according to the invention. This pharmaceutical is particularly provided for oral administration and is formulated, for example, as a syrup, emulsion, suspension or solution. In addition, the complex may be prepared as a drink suspension, a powder, chewable tablets, tablets for dispersion or dissolution or as effervescent tablet.

The invention further concerns the use of components a), b) and c) for the production of a non-bitter-tasting, water-insoluble complex.

The invention further comprises a method for the production of the non-bitter-tasting complex of active substance according to the invention, comprising the steps of:

i) providing a solution or a dispersion, which contains at least one bitter-tasting, active-substance molecule with at least one cationic group;

ii) providing a solution or dispersion, which contains at least one substance, an oligomer or/and a polymer which has at least one anionic group, and

iii) providing a substance, an oligomer or/and a polymer with least one cationic group and

iv) combining the components from (i), (ii), and (iii).

Preferably, the components from step (ii) and step (iii) are first mixed and then the solution or dispersion containing the active-substance molecule is added. The component of step (iii) can be provided as a solution or dispersion, but it is also possible to utilize this component as a solid substance.

In the procedure according to the invention, there is an intermixing of the components to form a spontaneous precipitate formation. This precipitate is insoluble in water and then can be used directly, for example, as a pharmaceutical.

The invention will be explained further by the following examples:

Materials

Carbopol 907 (linear polyacrylate, unbranched, BF Goodrich, Lot No. CC711BF812)

Carbopol 974 P NF (cross-linked polyacrylate with slow release in acidic medium, rapid release at high pH, BF Goodrich, Lot No. CC85AAB436)

Carbopol 971 P-NF (cross-linked polacrylate with slow release, BF Goodrich, Lot No. CC9NAAJ061)

10% HCl (Fluka)

Clarithromycin

Trimethylamine

Tetraphenylphosphonium bromide

10% Triethanolamine (Riedel-deHaen)

10% Acetic acid (Fluka)

PEG 1000 (Fluka)

Milli-Q water

Skim milk granulate (Reformhaus Diefenbach) Skim milk powder, Saliter, J. M. Gabler Saliter GmbH & Co KG, 87630 Obergünzburg, Allgäu, Lot No: LI900F046

### EXAMPLE 1

### Acceptance test of HMR 3647

HMR 3647 has the chemical formula C₄₃H₆₅N₅O₁₀ and has a molecular weight of 812.0 g/mol. It is a novel antibiotic from the group of ketolides, which is very poorly water-soluble and relatively lipophilic. At acidic pH, the solubility can be increased to 80 mg/ml, since the protonated cation is formed at this pH.

If tablets are applied for administration of the active substance, the bitter taste can be masked simply by an envelope. For application in children, however, a liquid application form is necessary, which will not be refused because of its bitter taste. The dose to be administered is, of course, dependent upon the body weight of the child and lies between 100 mg for children of one year and 1 g for patients who are 18 years old. Since the medication should be administered in an adequate volume, the concentration in the liquid application form should lie as far as possible between 25 and 50 mg/ml.

For conducting the acceptance test, HMR 3647 lyophilized product was dispersed in water and then adjusted to the desired concentration. Then the subjects each received 1 ml of the dispersion for one minute in the mouth. After the dispersion was spit out, crystals remaining in the mouth were checked for their bitter taste at a longer time point, after the dispersion was spit out. For comparison, the subjects were administered pure well water.

The acceptance test resulted in the fact that with oral application, the product was found unaccepable starting from a concentration of 2.5 μg/ml. Starting from a concentration of 10 μg/ml, all test persons found the taste unacceptable.

### EXAMPLE 2

### Investigations to determine the optimal precipitation conditions

Both of the above-named Carbopols are soluble in water. The combination containing HMR 3647 leads to the formation of a sticky, gum-type precipitate after lyophilization. When the precipitate is dispersed in water, the bitter taste is encountered just as previously. The pH of the gel lay between 2.7 and 3.2. A dependence on concentration or any difference between the two types could not be established.

The addition of HMR 3647 after neutralization of the Carbopol solution with triethanolamine (pH 6.3-7.2) and subsequent reduction of the pH to approximately 4 with 10% acetic acid led to a primarly fine-particle precipitate. With the addition of polyethylene glycol 1000, an influence on the precipitate could not be observed.

Since during the lyophilization a sedimenting of not only the precipitate occurred, but also of the residual components remaining in solution, a centrifuging was conducted at various pH values in order to obtain precipitate.

Carbopol 907 in combination with milk powder leads to a good precipitate, while Carbopol 974P NF tended to remain in solution as a highly viscious gel. Under the test conditions, therefore, it could be obtained only by lyophilization and not by precipitation. Carbopol 907 has proven to be the preferred compound under the test conditions.

Finally, two preferred ways for producing effective, non-bitter-tasting complexes were developed with the use of Carbopol 907 and the corresponding administration form was made available. These two ways are described in the following two examples. The results are presented in an overview in Tables 1 and 2.

### EXAMPLE 3

### Production of the precipitates according to Method a

150 ml of Carbopol 907 were dispersed in 60 ml of Milli-Q water. The pH value of the obtained dispersion amounted to 3.68. Then, 0.67 ml of 10% triethanolamine solution was added in order to adjust a pH of 7.00.

A second solution was prepared by disolving 100 mg of HMR 3647 in 1.23 ml of 0.1 M HCl and 0.77 ml of Milli-Q water. This solution was added dropwise to the first solution. After 1.5 ml had been added dropwise to the first solution, a solution of 200 mg of milk powder in 2 ml of Milli-Q water was simulaneously added dropwise. This led to a drift of the pH value to pH 6.6 and to the formation of a fine precipitate.

Now, by varying the portion, a total of 50 mg of Carbopol 907 and 50 mg of milk powder were slowly added while stirring to the obtained preparation. After 5 minutes of stirring, the precipitate (BS a) was sedimented by centrifuging at 15,000 rpm.

A pH of 4.5 was adjusted by adding 1.57 ml of 10% acetic acid to the supernatant and then stirring was conducted for another 5 minutes. This led to the opalescence of the dispersion and to the formation of another fine precipitate. The precipitate obtained in this way (BS b) was sedimented by centrifuging at 15,000 rpm. BS a, BS b and the remaining supernatant were then lyophilized. As BS a, only 3.8% of the originally added component could be obtained as a non-bitter-tasting precipitate. The charge of active substance of this precipitate was over 40%. In addition to the dispersion, the crystals themselves also displayed no bitter taste. In contrast, precipitate BS b showed a clearly bitter taste.

TABLE 1
Results for producing the complex according to Method a

| | Weight of | Efficiency | | | |
|---|---|---|---|---|---|
| | the | (% | Charge of | Charge of | |
| | lyophilized | lyophilized | HMR 3647 | HMR 3647 | |
| | product | product) | in mg | in % | Taste |
| Supernatant | 559.7 mg | 83.8% | 60.2 mg | 10.8% | Not testable |
| BS a | 25.4 mg | 3.8% | 10.5 mg | 41.3% | Not bitter |
| BS b | 83.0 mg | 12.4% | 19.7 mg | 23.7% | Bitter |

### EXAMPLE 4

### Production of the precipitates according to Method b

100 mg of Carbopol 907 were dissolved in 50 ml of Milli-Q water. The pH of this solution amounted to 3.84. 100 mg of milk powder were added, whereby no change of the pH value was determined.

A second solution was prepared by dissolving 100 mg of HMR 3647 in 1.23 ml of 0.1 M HCl and 0.77 ml of Milli-Q water. This solution was added by drops to the first suspension. A pH of 4.8 was established by this procedure. After approximately 2 minutes of stirring the formation of a fine-particle precipitate could be observed. Neutralization (pH 7.0) of the suspension led to the clarification of the supernatant. After another approximately 10 minutes of stirring, the precipitate was sedimented by centrifuging at 15,000 rpm. This precipitate was named BS 1. A pH of 4.5 was adjusted by adding 1.23 ml of 10% acetic acid to the supernatant. This led to opalescence and to the formation of another fine-particle precipitate after five minutes of stirring. The thus-obtained precipitate was also sedimented by centrifuging at 15,000 rpm and was named BS 2. BS 1, BS 2 and the remaining supernatant were lyophilized.

As BS 1, only 2.2% of the originally added components could be obtained in the form of a non-bitter-tasting precipitate. The charge of active substance of this precipitate was clearly greater than 40%. In contrast, 33.6% of the originally added components were obtained as a non-bitter-tasting precipitate as BS 2. The charge of active substance of this precipitate was almost 50%. In addition to the dispersions, the crystals of BS 1 and BS 2 also revealed no bitter taste.

TABLE 2
Results for the production of the complex according to Method b

| | Weight of | Efficiency | | | |
|---|---|---|---|---|---|
| | the | (% | Charge of | Charge of | |
| | lyophilized | lyophilized | HMR 3647 | HMR 3647 | |
| | product | product) | in mg | in % | Taste |
| Supernatant | 283.3 mg | 64.2% | 31.5 mg | 11.1% | Not tested |
| BS 1 | 9.5 mg | 2.2% | 4.7 mg | 47.4% | Not bitter |
| BS 2 | 148.4 mg | 33.6% | 72.7 mg | 49% | Not bitter |

### EXAMPLE 5

### Solubility Test

All of the precipitates obtained, thus the lyophilized samples BS a, BS b, BS 1 and BS 2 as well as the lyophilized supernatants could be dissolved in 2.5 ml of concentrated hydrochloric acid, with a clear solution being obtained. After 45 minutes, the standard samples were diluted and investigated spectrometrically at 263 nm in comparison to standard samples for their content of active substance (for results, see Tables 1 and 2).

### EXAMPLE 6

A) A stock solution of 1 g of clarithromycin in 7 ml of concentrated hydrochloric acid is prepared and brought to 50.0 ml with 1 N hydrochloric acid. 100 mg of Carbopol 907 are mixed with 50 ml of pure water and left to swell for 3 hours. 100 mg of milk powder are added to the clear solution, which produces turbidity. 5.00 ml of clarithromycin stock solution are added while stirring. This first leads to a clarification of the solution and then to a precipitation. This precipitate is obtained by centrifugation and is dried. The residue is not bitter.

B) 100 mg of Carbopol 971P-NF are mixed with 50 ml of pure water and left to swell for 3 hours. 100 mg of trimethylamine are added to the clear solution. The solution remains clear. When 5.00 ml of clarithromycin stock solution are added, a white precipitate is formed, which can be obtained by sedimentation or centrifuging. The supernatant solution is clear.

C) 100 mg of Carbopol 907 are mixed with 50 ml of pure water and left to swell for 3 hours. 100 mg of tetraphenylphosphonium bromide are added to the clear solution. The solution becomes cloudy. When 5.00 ml of clarithromycin stock solution are added, a temporary clarification of the solution occurs and then there is a white precipitate, which can be obtained by sedimentation or centrifuging. The supernatant is clear.

D) 100 mg of Carbopol 971P-NF are mixed with 50 ml of pure water and left to swell for 3 hours. 100 mg of tetraphenylphosphonium bromide are added to the clear solution. The solution becomes cloudy. When 5.00 ml of clarithromycin stock solution are added, a temporary clarification of the solution occurs and then a white precipitate is formed, which can be obtained by sedimentation or centrifuging. The supernatant solution is clear.

1. A water-insoluble, non-bitter-tasting complex, containing at least the following three compounds:
a) at least one bitter-tasting, active-substance molecule with at least one cationic group,
b) a substance, an oligomer or/and a polymer with at least one anionic group and
c) a substance, an oligomer or/and a polymer with at least one cationic group.

2. The complex according to claim 1, wherein the cationic group of the bitter active-substance molecule according to (a) is a protonatable amine.

3. The complex according to claim 1, wherein the active-substance molecule is an antibiotic.

4. The complex according to claim 3, wherein the antibiotic is erythromycin, clarithromycin, bufomedil or HMR 3647.

5. The complex according to claim 1, wherein the polymer of (b) comprises carboxymethylcellulose, Carbomers, Carbopols, or polyacrylic acid.

6. The complex according to claim 1, wherein the polymer of (c) is a micelle-forming polymer.

7. The complex according to claim 6, wherein the polymer of (c) comprises a protein or a peptide.

8. The complex according to claim 1, wherein the complex decomposes in concentrated hydrochloric acid.

9. The complex according to claim 1, wherein the complex is stable at pH 3-4 for at least 1 hour in water.

10. The complex according to claim 1, wherein the complex has a fraction of active substance of more than 40%.

11. A method for the production of a non-bitter-tasting complex of active substance, comprising the steps of:
(i) providing a solution or a dispersion, which contains at least one bitter-tasting, active substance molecule with at least one cationic group,
(ii) providing a solution or a dispersion, which contains at least one substance, an oligomer or/and a polymer, which has at least one anionic group,
(iii) providing a substance, an oligomer or/and a polymer with at least one cationic group, and
(iv) combining the components from (i), (ii) and (iii).
